(19) United States
(12) Patent Application Publication
(10) Pub. No.: US 2010/0268223 A1
(43) Pub. Date: Oct. 21, 2010

(54) Methods for Image Analysis and Visualization of Medical Image Data Suitable for Use in Assessing Tissue Ablation and Systems and Methods for Controlling Tissue Ablation Using Same

(75) Inventors: Jonathan A. Coe, Denver, CO (US); Casey M. Latdkow, Westminster, CO (US)

Correspondence Address: TYCO Healthcare Group LP;Attn: IP Legal, 5920 Longbow Drive, Mail Stop A36, Boulder, CO, 80301-3299, US

(73) Assignee: TYCO Health Group LP

(21) Appl. No.: 12/819,330

(22) Filed: Jun. 21, 2010

Related U.S. Application Data
(63) Continuation-in-part of application No. 12/761,267, filed on Apr. 15, 2010.
(60) Provisional application No. 61/169,556, filed on Apr. 15, 2009.

(51) Int. Cl. A61B 18/14; G06K 9/34

(52) U.S. Cl. 606/41; 382/173

(57) ABSTRACT

A method of image analysis includes the initial step of receiving a data set including image data. The image data represents a sequence of 2-D slice images. The method includes the steps of segmenting an object of interest from surrounding image data of each slice image based on a p-value of a t-statistic relating each pixel successively examined to statistical properties derived from pixel values within the region of interest, and rendering a volume of the object of interest using (x,y) coordinates corresponding to boundaries of the segmented object of interest.

### CROSS-REFERENCE TO RELATED APPLICATION

The present application is a continuation-in-part application, which claims priority to, and the benefit of, U.S. patent application Ser. No. 12/761,267 filed on Apr. 15, 2010, which claims priority to, and the benefit of, U.S. Provisional Application Ser. No. 61/169,556 filed on Apr. 15, 2009, the disclosures of which are herein incorporated by reference in their entireties.

### BACKGROUND

1. Technical Field

The present disclosure relates to data analysis and visualization techniques, and, more particularly, to methods for image analysis and visualization of medical image data that are suitable for use in assessing biological tissue ablation, and systems and methods for controlling tissue ablation using the same.

2. Discussion of Related Art

Treatment of certain diseases requires the destruction of malignant tissue growths, e.g., tumors. Electromagnetic radiation can be used to heat and destroy tumor cells. Treatment may involve inserting ablation probes into tissues where cancerous tumors have been identified. Once the probes are positioned, electromagnetic energy is passed through the probes into surrounding tissue.

In the treatment of diseases such as cancer, certain types of tumor cells have been found to denature at elevated temperatures that are slightly lower than temperatures normally injurious to healthy cells. Known treatment methods, such as hyperthermia therapy, heat diseased cells to temperatures above 41° C. while maintaining adjacent healthy cells below the temperature at which irreversible cell destruction occurs. These methods involve applying electromagnetic radiation to heat, ablate and/or coagulate tissue. Microwave energy is sometimes utilized to perform these methods. Other procedures utilizing electromagnetic radiation to heat tissue also include coagulation, cutting and/or ablation of tissue. Many procedures and types of devices utilizing electromagnetic radiation to heat tissue have been developed.

Medical imaging has become a significant component in the clinical setting and in basic physiology and biology research, e.g., due to enhanced spatial resolution, accuracy and contrast mechanisms that have been made widely available. Medical imaging now incorporates a wide variety of modalities that noninvasively capture the structure and function of the human body. Such images are acquired and used in many different ways including medical images for diagnosis, staging and therapeutic management of malignant disease.

Because of their anatomic detail, computed tomography (CT) and magnetic resonance imaging (MRI) are suitable for, among other things, evaluating the proximity of tumors to local structures. CT and MRI scans produce two-dimensional (2-D) axial images, or slices, of the body that may be viewed sequentially by radiologists who visualize or extrapolate from these views actual three-dimensional (3-D) anatomy.

Medical image processing, analysis and visualization play an increasingly significant role in many fields of biomedical research and clinical practice. While images of modalities such as MRI or CT may be displayed as 2-D slices, three-dimensional visualization of images and quantitative analysis requires explicitly defined object boundaries. For example, to generate a 3-D rendering of a tumor from a MRI image, the tumor needs to be first identified within the image and then the tumor's boundary marked and used for 3-D rendering. Measurements and quantitative analysis for parameters such as area, perimeter, volume and length may be obtained when object boundaries are defined.

A boundary in an image is a contour that represents the change from one object or surface to another. Image segmentation involves finding salient regions and their boundaries. A number of image segmentation methods have been developed using fully automatic or semi-automatic approaches for medical imaging and other applications. Medical image segmentation refers to the delineation of anatomical structures and other regions of interest in medical images for assisting doctors in evaluating medical imagery or in recognizing abnormal findings in a medical image. Structures of interest may include organs or parts thereof, such as cardiac ventricles or kidneys, abnormalities such as tumors and cysts, as well as other structures such as bones and vessels. Despite the existence of numerous image segmentation techniques, segmentation of medical images is still a challenge due to the variety and complexity of medical images.

Medical image analysis and visualization play an increasingly significant role in disease diagnosis and monitoring as well as, among other things, surgical planning and monitoring of therapeutic procedures. Three-dimensional image visualization techniques may be used to provide the clinician with a more complete view of the anatomy, reducing the variability of conventional 2-D visualization techniques. Three-dimensional visualization of medical images of modalities such as CT or MRI may facilitate planning and effective execution of therapeutic hyperthermic treatments.

### SUMMARY

The present disclosure relates to a method of image analysis including the initial step of receiving a data set including image data. The image data represents a sequence of two-dimensional (2-D) slice images. The method includes the steps of segmenting an object of interest from surrounding image data of each slice image based on a p-value of a t-statistic relating each pixel successively examined to statistical properties derived from pixel values within the region of interest, and rendering a volume of the object of interest using (x,y) coordinates corresponding to boundaries of the segmented object of interest.

The present disclosure relates to a method of image analysis including the initial step of receiving a data set including image data. The image data represents a sequence of 2-D slice images. The method includes the steps of selectively defining a region of interest within each slice image of the sequence of 2-D slice images, characterizing pixels contained within the region of interest of each slice image based on statistical properties derived from pixel values within the region of interest of each slice image, and segmenting an object of interest from surrounding image data of each slice image based on a p-value of a t-statistic relating each pixel successively examined to statistical properties derived from pixel values within the region of interest. The method also includes the steps of determining (x,y) coordinates corresponding to boundaries of the segmented object of interest of each slice image, and rendering a volume of the object of interest using the (x,y) coordinates.

The present disclosure also relates to an electrosurgical system including an electrosurgical power generating source and an energy-delivery device operably associated with the electrosurgical power generating source. The electrosurgical system also includes a processor unit and an imaging system capable of generating image data representing a sequence of 2-D slice images. The processor unit is disposed in operative communication with the imaging system and adapted to analyze the image data to segment an object of interest from surrounding image data of each slice image based on a p-value of a t-statistic relating each pixel successively examined to statistical properties derived from pixel values within the region of interest.

### BRIEF DESCRIPTION OF THE DRAWINGS

Objects and features of the presently disclosed methods for image analysis and visualization of medical image data and the presently disclosed systems and methods for controlling tissue ablation using the same will become apparent to those of ordinary skill in the art when descriptions of various embodiments thereof are read with reference to the accompanying drawings, of which:

FIG. 1 is a schematic illustration of an ablation system including an energy applicator positioned for the delivery of energy to a targeted tissue area according to an embodiment of the present disclosure;

FIG. 2 is a diagrammatic representation of a two-dimensional (2-D) image slice showing patient tissue surrounding an object of interest according to an embodiment of the present disclosure;

FIG. 3 is a diagrammatic representation of the 2-D image slice of FIG. 2 showing a user-defined region of interest shown by a dashed circle within the object of interest according to an embodiment of the present disclosure;

FIG. 4 is a diagrammatic representation of a thresholded image of the 2-D image slice of FIG. 2 according to an embodiment of the present disclosure;

FIG. 5 is a diagrammatic representation of a resulting image of topographical rule based processing showing the segmented object of interest of FIG. 4 according to an embodiment of the present disclosure;

FIGS. 6A and 6B are diagrammatic representations of morphological dilation and erosion operations on the object of interest of FIG. 5 according to an embodiment of the present disclosure;

FIG. 7 is a schematic view of a volume-rendered ablation according to an embodiment of the present disclosure;

FIG. 8 is a schematic view of a volume-rendered ablation according to an embodiment of the present disclosure;

FIG. 9 is a flowchart illustrating a method of image analysis according to an embodiment of the present disclosure;

FIG. 10 is a flowchart illustrating a method of directing energy to tissue according to an embodiment of the present disclosure;

FIG. 11 is a diagrammatic representation of a 2-D image slice showing a medium surrounding an object of interest according to an embodiment of the present disclosure;

FIGS. 12 through 14 are diagrammatic representations showing sequentially-illustrated, region-growing operations on a region of interest within the object of interest of FIG. 11 in accordance with the present disclosure;

FIG. 15 is a diagrammatic representation showing the J^{th} iteration of a region-growing method on the growing region of interest of FIG. 14 in accordance with the present disclosure;

FIG. 16 is a diagrammatic representation showing the K^{th} iteration of a region-growing method on the growing region of interest of FIG. 15 in accordance with the present disclosure;

FIG. 17 is a diagrammatic representation showing the L^{th} iteration of a region-growing method on the growing region of interest of FIG. 16 in accordance with the present disclosure;

FIG. 18 is a diagrammatic representation showing the thresholded, region-grown region of interest of FIG. 17 according to an embodiment of the present disclosure;

FIG. 19 is a schematic diagram of an ablation system including an electrosurgical device according to an embodiment of the present disclosure;

FIG. 20 is a schematically-illustrated representation of simulation results showing a broadside radiation pattern according to an embodiment of the present disclosure;

FIG. 21 is a flowchart illustrating another embodiment of a method of image analysis in accordance with the present disclosure; and

FIG. 22 is a flowchart illustrating yet another embodiment of a method of image analysis in accordance with the present disclosure.

### DETAILED DESCRIPTION

Hereinafter, embodiments of presently disclosed methods for image analysis and visualization of medical image data and the presently disclosed systems and methods for controlling tissue ablation using the same are described with reference to the accompanying drawings. Like reference numerals may refer to similar or identical elements throughout the description of the figures. As shown in the drawings and as used in this description, and as is traditional when referring to relative positioning on an object, the term “proximal” refers to that portion of the object that is closer to the user and the term “distal” refers to that portion of the object that is farther from the user.

This description may use the phrases “in an embodiment,” “in embodiments,” “in some embodiments,” or “in other embodiments,” which may each refer to one or more of the same or different embodiments in accordance with the present disclosure. For the purposes of this description, a phrase in the form “A/B” means A or B. For the purposes of the description, a phrase in the form “A and/or B” means “(A), (B), or (A and B)”. For the purposes of this description, a phrase in the form “at least one of A, B, or C” means “(A), (B), (C), (A and B), (A and C), (B and C), or (A, B and C)”.

Electromagnetic energy is generally classified by increasing energy or decreasing wavelength into radio waves, microwaves, infrared, visible light, ultraviolet, X-rays and gamma-rays. As it is used in this description, “microwave” generally refers to electromagnetic waves in the frequency range of 300 megahertz (MHz) (3×10⁸ cycles/second) to 300 gigahertz (GHz) (3×10¹¹ cycles/second). As it is used in this description, “ablation procedure” generally refers to any ablation procedure, such as microwave ablation, radio frequency (RF) ablation or microwave ablation assisted resection. As it is used in this description, “energy applicator” generally refers to any device that can be used to transfer energy from a power generating source, such as a microwave or RF electrosurgical generator, to tissue. As it is used in this description, “transmission line” generally refers to any transmission medium that can be used for the propagation of signals from one point to another.

As it is used in this description, “length” may refer to electrical length or physical length. In general, electrical length is an expression of the length of a transmission medium in terms of the wavelength of a signal propagating within the medium. Electrical length is normally expressed in terms of wavelength, radians or degrees. For example, electrical length may be expressed as a multiple or sub-multiple of the wavelength of an electromagnetic wave or electrical signal propagating within a transmission medium. The wavelength may be expressed in radians or in artificial units of angular measure, such as degrees. The electric length of a transmission medium may be expressed as its physical length multiplied by the ratio of (a) the propagation time of an electrical or electromagnetic signal through the medium to (b) the propagation time of an electromagnetic wave in free space over a distance equal to the physical length of the medium. The electrical length is in general different from the physical length. By the addition of an appropriate reactive element (capacitive or inductive), the electrical length may be made significantly shorter or longer than the physical length.

As used in this description, the term “real-time” means generally with no observable latency between data processing and display. As used in this description, “near real-time” generally refers to a relatively short time span between the time of data acquisition and display.

Various embodiments of the present disclosure provide systems and methods of directing energy to tissue. Embodiments may be implemented using electromagnetic radiation at microwave frequencies or at other frequencies. An electromagnetic energy delivery device including an energy applicator array, according to various embodiments, is designed and configured to operate between about 300 MHz and about 10 GHz.

Various embodiments of the presently disclosed electrosurgical system including an energy applicator, or energy applicator array, are suitable for microwave ablation and for use to pre-coagulate tissue for microwave ablation assisted surgical resection. In addition, although the following description describes the use of a dipole microwave antenna, the teachings of the present disclosure may also apply to a monopole, helical, or other suitable type of microwave antenna.

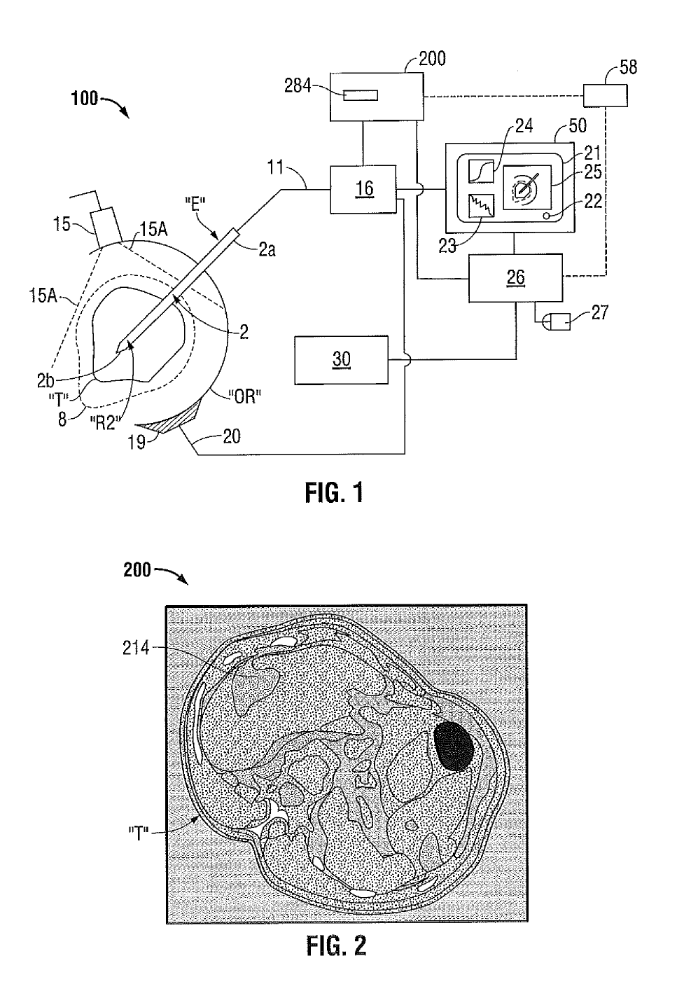

An electrosurgical system 100 according to an embodiment of the present disclosure is shown in FIG. 1 and includes an electromagnetic energy delivery device or energy applicator array “E”. In some embodiments, the energy-delivery device is configured to emit a directional radiation pattern that rotates therewith during rotation of the energy-delivery device about the longitudinal axis thereof. Energy applicator array “E” includes an energy applicator or probe 2. As one of ordinary skill in the art will readily recognize, other energy applicator array “E” embodiments may include a plurality of energy applicators.

Probe 2 generally includes a radiating section “R2” operably connected by a feedline (or shaft) 2a to an electrosurgical power generating source 16, e.g., a microwave or RF electrosurgical generator. In some embodiments, the power generating source 28 is configured to provide microwave energy at an operational frequency from about 300 MHz to about 10 GHz. Power generating source 16 may be configured to provide various frequencies of electromagnetic energy. A transmission line 11 may be provided to electrically couple the feedline 2a to the electrosurgical power generating source 16.

Feedline 2a may be formed from a suitable flexible, semi-rigid or rigid microwave conductive cable, and may connect directly to an electrosurgical power generating source 16. Feedline 2a may have a variable length from a proximal end of the radiating section “R2” to a distal end of the transmission line 11 ranging from a length of about one inch to about twelve inches. Transmission line 11 may additionally, or alternatively, provide a conduit (not shown) configured to provide coolant fluid from a coolant source (not shown) to the energy applicator array “E”.

Located at the distal end of the probe 2 is a tip portion 2b, which may be configured to be inserted into an organ “OR” of a human body or any other body tissue. As it is used in this description, “organ” may refer to any anatomical organ or region of interest. Tip portion 2b may terminate in a sharp tip to allow for insertion into tissue with minimal resistance. Tip portion 2b may include other shapes, such as, for example, a tip that is rounded, flat, square, hexagonal, or cylindroconical.

Electrosurgical system 100 includes a user interface 50. User interface 50 may include a display device 21, such as without limitation a flat panel graphic LCD (liquid crystal display), adapted to visually display one or more user interface elements 23, 24, 25. In an embodiment, the display device 21 includes touchscreen capability (not shown), e.g., the ability to receive input from an object in physical contact with the display device 21, such as without limitation a stylus or a user's fingertip. A user interface element 23, 24, 25 may have a corresponding active region, such that, by touching the display panel within the active region associated with the user interface element, an input associated with the user interface element 23, 24, 25 is received by the user interface 50.

User interface 50 may additionally, or alternatively, include one or more controls 22 that may include without limitation a switch (e.g., pushbutton switch, toggle switch, slide switch) and/or a continuous actuator (e.g., rotary or linear potentiometer, rotary or linear encoder). In an embodiment, a control 22 has a dedicated function, e.g., display contrast, power on/off, and the like. Control 22 may also have a function that may vary in accordance with an operational mode of the electrosurgical system 100. A user interface element (e.g., 23 shown in FIG. 1) may be provided to indicate the function of the control 22. Control 22 may also include an indicator, such as an illuminated indicator, e.g., a single- or variably-colored LED (light emitting diode) indicator.

As shown in FIG. 1, the electrosurgical system 100 may include a reference electrode 19 (also referred to herein as a “return” electrode). Return electrode 19 may be electrically coupled via a transmission line 20 to the power generating source 16. During a procedure, the return electrode 19 may be positioned in contact with the skin of the patient or a surface of the organ “OR”. When the surgeon activates the energy applicator array “E”, the return electrode 19 and the transmission line 20 may serve as a return current path for the current flowing from the power generating source 16 through the probe 2.

During microwave ablation using the electrosurgical system 100 the energy applicator array “E” is inserted into or placed adjacent to tissue and microwave energy is supplied thereto. Ultrasound or computed tomography (CT) guidance may be used to accurately guide the energy applicator array “E” into the area of tissue to be treated. Probe 2 may be placed percutaneously or surgically, e.g., using conventional surgical techniques by surgical staff. A clinician may pre-determine the length of time that microwave energy is to be applied. Application duration may depend on a variety of factors such as energy applicator design, number of energy applicators used simultaneously, tumor size and location, and whether the tumor was a secondary or primary cancer. The duration of microwave energy application using the energy applicator array “E” may depend on the progress of the heat distribution within the tissue area that is to be destroyed and/or the surrounding tissue.

FIG. 1 shows a targeted region including ablation targeted tissue represented in sectional view by the solid line “T”. It may be desirable to ablate the targeted region “T” by fully engulfing the targeted region “T” in a volume of lethal heat elevation. Targeted region “T” may be, for example, a tumor that has been detected by a medical imaging system 30.

Medical imaging system 30, according to various embodiments, includes a scanner (e.g., 15 shown in FIG. 1) of any suitable imaging modality, or other image acquisition device capable of generating input pixel data representative of an image. Medical imaging system 30 may additionally, or alternatively, include a medical imager operable to form a visible representation of the image based on the input pixel data. Medical imaging system 30 may include a storage device such as an internal memory unit, which may include an internal memory card and removable memory. In some embodiments, the medical imaging system 30 may be a multi-modal imaging system capable of scanning using different modalities. Examples of imaging modalities that may be suitably and selectively used include X-ray systems, ultrasound (UT) systems, magnetic resonance imaging (MRI) systems, computed tomography (CT) systems, single photon emission computed tomography (SPECT), and positron emission tomography (PET) systems, each of which may generate image information according to a different protocol. Medical imaging system 30 may include any device capable of generating digital data representing an anatomical region of interest. In some embodiments, the medical imaging system 30 includes a MRI scanner and/or a CT scanner capable of generating two-dimensional (2-D) image slices (e.g., 200 shown in FIG. 2).

Image data representative of one or more images may be communicated between the medical imaging system 30 and a processor unit 26. Medical imaging system 30 and the processor unit 26 may utilize wired communication and/or wireless communication. Processor unit 26 may include any type of computing device, computational circuit, or any type of processor or processing circuit capable of executing a series of instructions that are stored in a memory (not shown) associated with the processor unit 26. Processor unit 26 may be adapted to run an operating system platform and application programs. Processor unit 26 may receive user inputs from a keyboard (not shown), a pointing device 27, e.g., a mouse, joystick or trackball, and/or other device communicatively coupled to the processor unit 26.

According to embodiments of the present disclosure, the processor unit 26 is operably associated with an electrosurgical power generating source (e.g., 16 shown in FIG. 1) and adapted to determine one or more operating parameters associated with the electrosurgical power generating source based on one or more parameters of a volume-rendered object of interest. Examples of operating parameters associated with the electrosurgical power generating source include without limitation temperature, impedance, power, current, voltage, mode of operation, and duration of application of electromagnetic energy.

A scanner (e.g., 15 shown in FIG. 1) of any suitable imaging modality may additionally, or alternatively, be disposed in contact with the organ “OR” to provide image data. As an illustrative example, the two dashed lines 15A in FIG. 1 bound a region for examination by the scanner 15, e.g., a CT scanner.

In FIG. 1, the dashed line 8 surrounding the targeted region “T” represents the ablation isotherm in a sectional view through the organ “OR”. The shape and size of the ablation volume, as illustrated by the dashed line 8, may be influenced by a variety of factors including the configuration of the energy applicator array “E”, the geometry of the radiating section “R2”, cooling of the probe 2, ablation time and wattage, and tissue characteristics, e.g., impedance. Processor unit 26 may be connected to one or more display devices (e.g., 21 shown in FIG. 1) for displaying output from the processor unit 26, which may be used by the clinician to visualize the targeted region “T” and/or the ablation volume 8 and/or a volume-rendered ablation (e.g., 700 shown in FIG. 7) in real-time or near real-time during a procedure, e.g., an ablation procedure. In some embodiments, the patient's anatomy may be scanned by one or more of several scanning modalities, such as CT scanning, MRI scanning, ultrasound, and/or PET scanning, e.g., to visualize a tumor and the surrounding normal tissue. The tumor dimensions may thereby be determined and/or the location of the tumor relative to critical structures and the external anatomy may be ascertained.

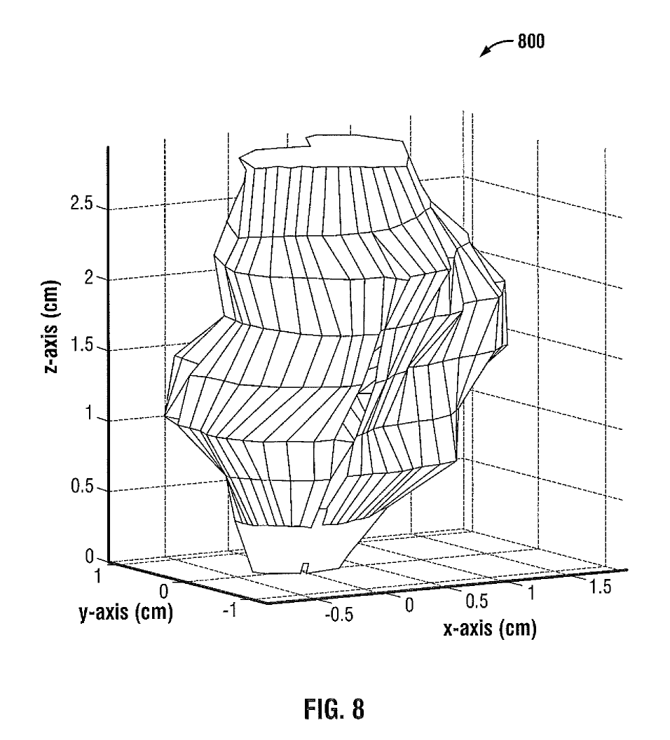

Electrosurgical system 100 may include a library 200. As it is used in this description, “library” generally refers to any repository, databank, database, cache, storage unit and the like. Library 200 may include a database 284 that is configured to store and retrieve energy applicator data, e.g., parameters associated with one or energy applicators and/or one or more energy applicator arrays. Parameters stored in the database 284 in connection with an energy applicator array may include, but are not limited to, energy applicator array identifier, energy applicator array dimensions, a frequency, an ablation length, an ablation diameter, a temporal coefficient, a shape metric, and/or a frequency metric. Volume-rendered ablations (e.g., 700 and 800 shown in FIGS. 7 and 8, respectively) may be stored in the database 284. In an embodiment, ablation pattern topology may be included in the database 284, e.g., a wireframe model of an energy applicator array (e.g., 25 shown in FIG. 1) and/or a representation of a radiation pattern associated therewith.

Library 200 according to embodiments of the present disclosure may be communicatively associated with a picture archiving and communication system (PACS) database (shown generally as 58 in FIG. 1) containing DICOM (acronym for Digital Imaging and Communications in Medicine) formatted medical images. PACS database 58 may be configured to store and retrieve image data, e.g., representing a sequence of two-dimensional (2-D) slice images, from a variety of imaging modalities. As shown in FIG. 1, the processor unit 26 may be communicatively associated with the PACS database 58. In accordance with one or more presently-disclosed methods, image data associated with a prior treatment of a target tissue volume is retrieved from the PACS database 58 and the ablation volume is rendered using a sequence of 2-D image slices of the image data.

Images and/or non-graphical data stored in the library 200, and/or retrievable from the PACS database 58, may be used to configure the electrosurgical system 100 and/or control operations thereof. For example, volume-rendered ablations (e.g., 700 and 800 shown in FIGS. 7 and 8, respectively) associated with an energy applicator, according to embodiments of the present disclosure, may be used as a feedback tool to control an instrument's and/or clinician's motion, e.g., to allow clinicians to avoid ablating critical structures, such as large vessels, healthy organs or vital membrane barriers.

Images and/or non-graphical data stored in the library 200, and/or retrievable from the PACS database 58, such as volume-rendered ablations (e.g., 700 and 800 shown in FIGS. 7 and 8, respectively) may be used to facilitate planning and effective execution of a procedure, e.g., an ablation procedure. Images and/or information displayed on the display device 21 of the user interface 50, for example, may be used by the clinician to better visualize and understand how to achieve more optimized results during thermal treatment of tissue, such as, for example, ablation of tissue, tumors and cancer cells. One or more parameters of volume-rendered ablations according to embodiments of the present disclosure may be used to determine one or more operating parameters associated with an electrosurgical power generating source, inter-operatively and/or pre-operatively.

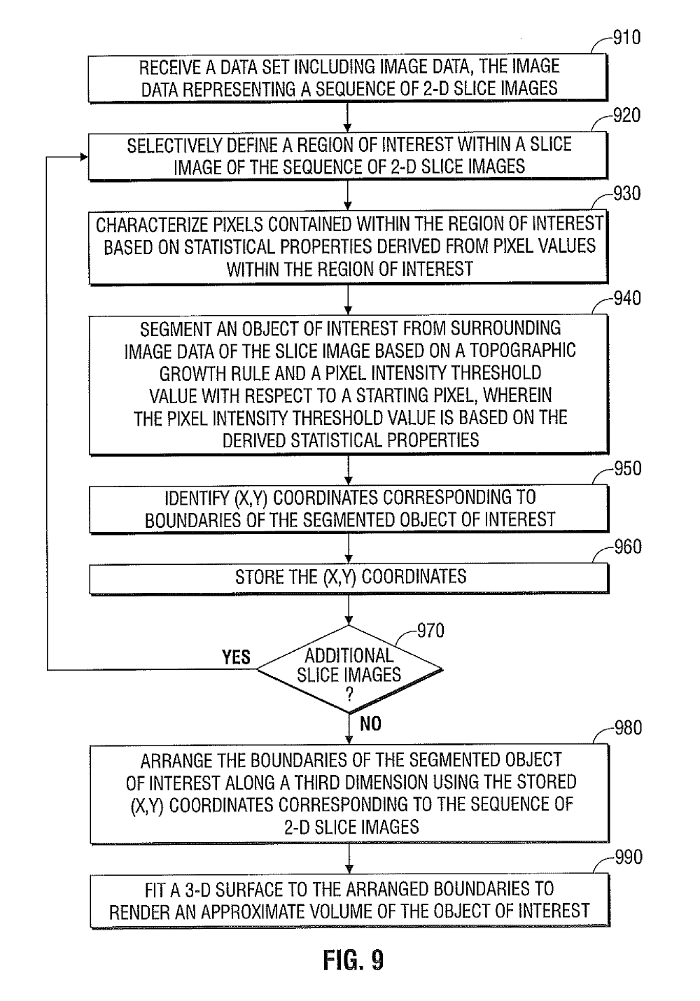

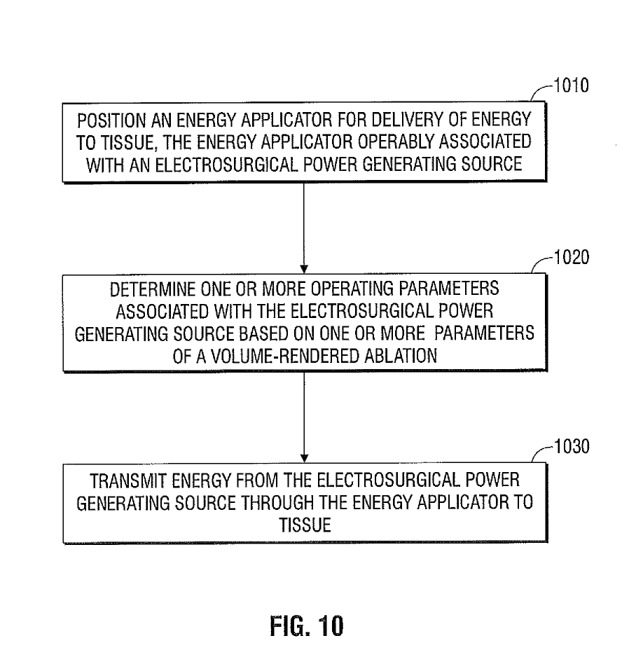

Hereinafter, methods of image analysis are described with reference to FIGS. 9, 21 and 22, and a method of directing energy to tissue is described with reference to FIG. 10. It is to be understood that the steps of the methods provided herein may be performed in combination and in a different order than presented herein without departing from the scope of the disclosure.

FIG. 9 is a flowchart illustrating a method of image analysis according to an embodiment of the present disclosure. In step 910, a data set including image data is received. The image data represents a sequence of 2-D slice images. The data set may include DICOM format images of any part of the body or a full-body scan. However, it will be appreciated that the data set may include image and/or patient data in any standard format, such as without limitation DICOS (Digital Imaging and Communication in Security) format, DICONDE (Digital Imaging and Communication in Nondestructive Evaluation) format, or other format which may include a file format definition and a network communications protocol. The image data may include inter-operatively acquired images and/or pre-operatively acquired images. A subset of the image data may be selectively identified for processing in accordance with the method of image analysis illustrated in FIG. 9.

In step 920, a region of interest is selectively defined within a slice image (e.g., 200 shown in FIG. 2) of the sequence of 2-D slice images. In FIG. 3, the dashed circle 311 is a user-defined region of interest. Referring to FIG. 1, the clinician may use a pointing device 27 coupled to the processor unit 26 and/or the touchscreen capability of the display device 21 of the electrosurgical system 100 to create a circle, or other shape, around a selected region of interest (e.g., 310 shown in FIG. 3) within an object of interest (e.g., 214 shown in FIGS. 2 and 3).

In step 930, pixels contained within the region of interest are characterized based on statistical properties derived from pixel values within the region of interest. Pixel values in a grayscale image define gray levels (or shades of gray). For example, grayscale images may be stored with 16 bits per pixel, which allows 65,536 grey levels to be recorded. In embodiments, the mean pixel grey-level values and their standard deviations are measured within the region of interest (e.g., 310 shown in FIG. 3). In embodiments, the pixel intensity threshold is any pixel in the entire image that is within a mean value of the region-of-interest gray levels+/−a predetermined multiple of the standard deviation of the region-of-interest gray levels. In one embodiment, the predetermined multiple of the standard deviation is 1.8.

In step 940, an object of interest (e.g., 214 shown in FIGS. 2 and 3) is segmented from the surrounding image data based on a topographic growth rule and a pixel intensity threshold value with respect to a starting pixel. The pixel intensity threshold value is based on the statistical properties derived in step 930. The starting pixel (e.g., 312 shown in FIG. 3) may be automatically selected, e.g., using knowledge of an anatomical structure or a region of interest. The starting pixel may be user-defined, and may be a pixel located at or near the center of a user-defined shape element, e.g., the dashed circle 311 shown in FIG. 3.

In some embodiments, a plurality of starting pixels (also referred to herein as seed pixels) may be selectively defined. In one variation, one or more seed pixels may be selected from among the pixels associated with a user-defined shape element, e.g., the circle 1105 shown in FIG. 11.

A method of region growing, according to an embodiment of the present disclosure, is used basically to select pixels that are within a certain range of the starting pixel value. A topographic growth rule is applied requiring that the pixels are within a given template, e.g., adjacency, connectivity, or containment of contiguous features. Pixel intensity thresholding is used to identify pixels that are within a certain grey-level value with respect to a starting pixel and/or with respect to a certain average or mean value, e.g., mean value of the region-of-interest gray levels. In some embodiments, the pixel adjacent relationship may be 4-connectivity (vertical, horizontal). In other embodiments, an 8-connected neighborhood may be chosen for the pixels adjacent relationship.

Starting at the starting pixel (e.g., 312 shown in FIG. 3), the presently disclosed method of region growing proceeds to iteratively examine adjacent pixels, and pixels that are within a predetermined grey-level value of each other are appended to the growing region of interest (also referred to herein as the processed entity). In some embodiments, the value of the predetermined grey-level value is in the range of about 0 to about 500. Typically, but not necessarily, the grey-level value 0 corresponds to black.

Through the successive iterations of the presently disclosed region-growing method, the analysis of the mean and the standard deviation of the region-of-interest gray levels are effected to join or not join the examined pixels to the processed entity.

Image analysis methods according to embodiments of the present disclosure may include thresholding to segment image data by setting all pixels whose intensity values are above a predetermined threshold to a foreground value and all the remaining pixels to a background value. Thresholding may produce a segmentation that yields substantially all the pixels that belong to the object of interest in the image data. Thresholding may be applied to an entire image, or may be used on a region by region basis.

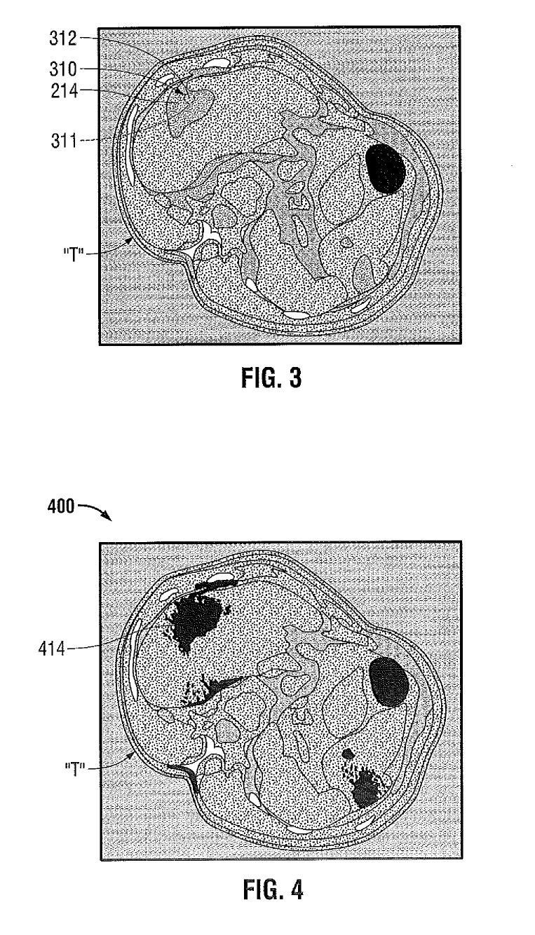

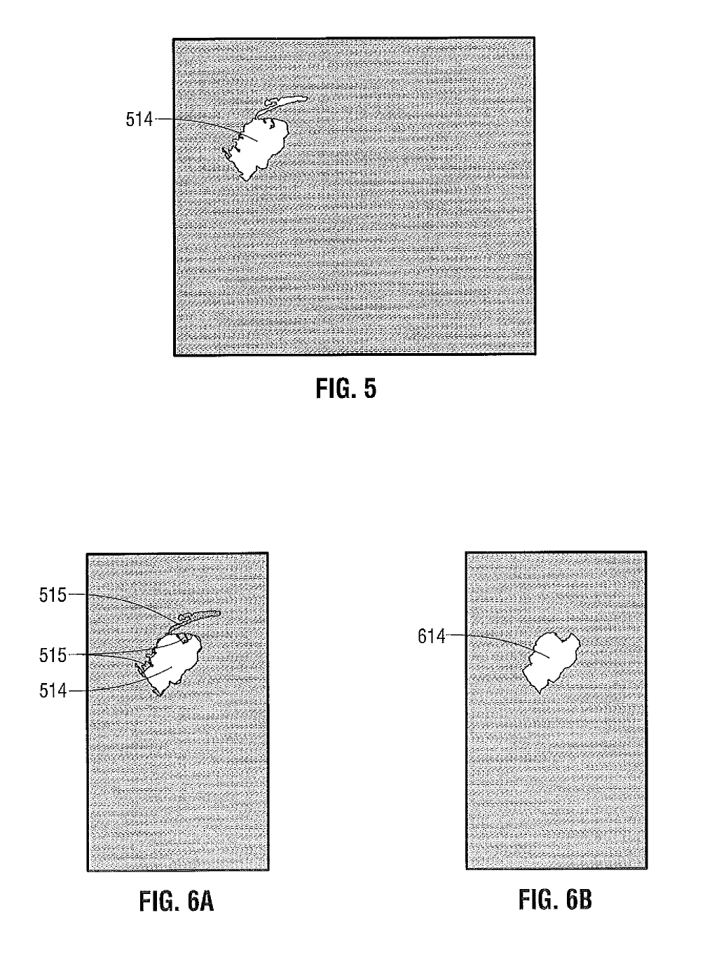

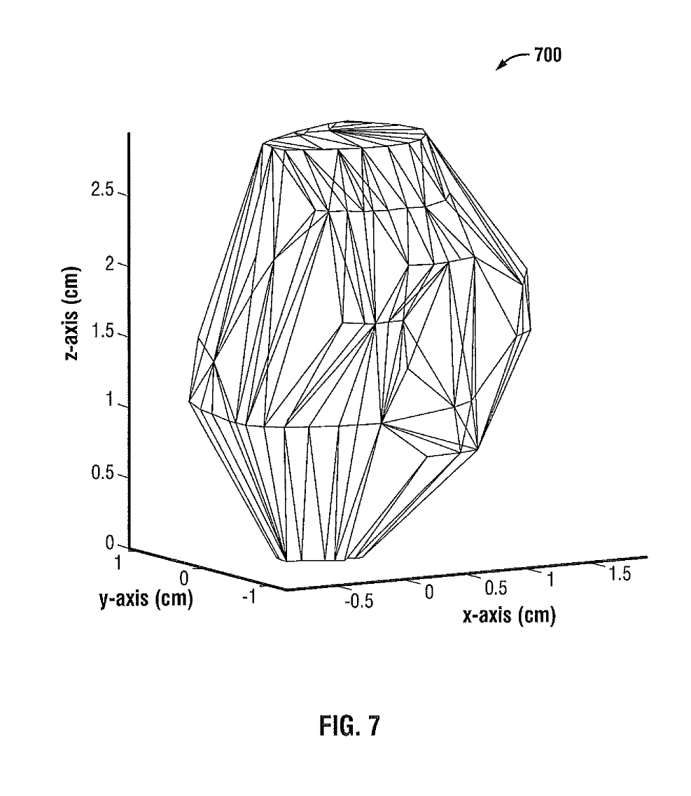

FIG. 4 shows a thresholded 2-D image slice that includes a thresholded object of interest 414 (e.g., an ablation) according to an embodiment of the present disclosure. FIG. 5 shows the segmented object of interest 514 resulting from topographical rule-based processing of the region of interest of FIG. 4 according to an embodiment of the present disclosure. FIGS. 6A and 6B show an object of interest 614 resulting from morphological dilation and erosion operations on the object of interest 514 of FIG. 5 that discarded stringers 515 according to an embodiment of the present disclosure.

In step 950, (x,y) coordinates corresponding to the boundaries of the segmented object of interest are determined. The (x,y) coordinates are stored in a memory, in step 960.

NON In step 970, it is determined whether additional slice images remain to be processed. If it is determined that there are additional slice images, then the method repeats step 920 through step 960, as described above.

If it is determined that there are no additional slice images, then, in step 980, the boundaries of the segmented object of interest are arranged along a third dimension using the stored (x,y) coordinates associated with the plurality of 2-D slice images. The spacing of this arrangement along the third dimension (e.g., z-axis shown in FIGS. 7 and 8) is preferably equal to the anatomic spacing of the medical images.

In step 990, a 3-D surface is fitted to the arranged boundaries to render an approximate volume of the object of interest. For this purpose, least-squares fitting (regression), or other methods, may be used. In some embodiments, quantitative analysis may be performed for determining the size, density and other parameters of the volume-rendered object of interest. Data associated with the object of interest (e.g., 700 and 800 shown in FIGS. 7 and 8, respectively) may be stored in a database (e.g., 284 shown in FIG. 1), and may be used for controlling an ablation procedure. For example, one or more operating parameters associated with an electrosurgical power generating source may be determined based one or more parameters of a volume-rendered ablation.

FIG. 10 is a flowchart illustrating a method of directing energy to tissue according to an embodiment of the present disclosure. In step 1010, an energy applicator (e.g., “E” shown in FIG. 1) is positioned for delivery of energy to tissue (e.g., “T” shown in FIG. 1), wherein the energy applicator is operably associated with an electrosurgical power generating source (e.g., 16 shown in FIG. 1).

In step 1020, one or more operating parameters associated with the electrosurgical power generating source are determined based one or more parameters of a volume-rendered ablation. Examples of operating parameters associated with the electrosurgical power generating source include without limitation temperature, impedance, power, current, voltage, mode of operation, and duration of application of electromagnetic energy. Examples of parameters of a volume-rendered ablation (e.g., 700 and 800 shown in FIGS. 7 and 8, respectively) include without limitation volume, length, diameter, minimum diameter, maximum diameter and centroid. Volume-rendered ablations may be stored in a database (e.g., 284 shown in FIG. 1) prior to and/or during a procedure. In embodiments, the volume-rendered ablation is generated from image data representing a sequence of 2-D slice images, e.g., in accordance with the presently disclosed image analysis method illustrated in FIG. 9.

In step 1030, energy from the electrosurgical power generating source is transmitted through the energy applicator to tissue. The duration of energy application using the energy applicator may depend on the progress of the heat distribution within the tissue area that is to be destroyed and/or the surrounding tissue. In some embodiments, the duration of energy application using the energy applicator may depend on, among other things, the volume of a volume-rendered ablation, e.g., stored in a database (e.g., PACS database 58 shown in FIG. 1).

FIGS. 12 through 14 show sequentially-illustrated, region-growing operations on a region of interest “R₁” located within an object of interest 1114, which is surrounded by a medium “M”, e.g., tissue, of a 2-D image slice (shown generally as 1100 in FIG. 11).

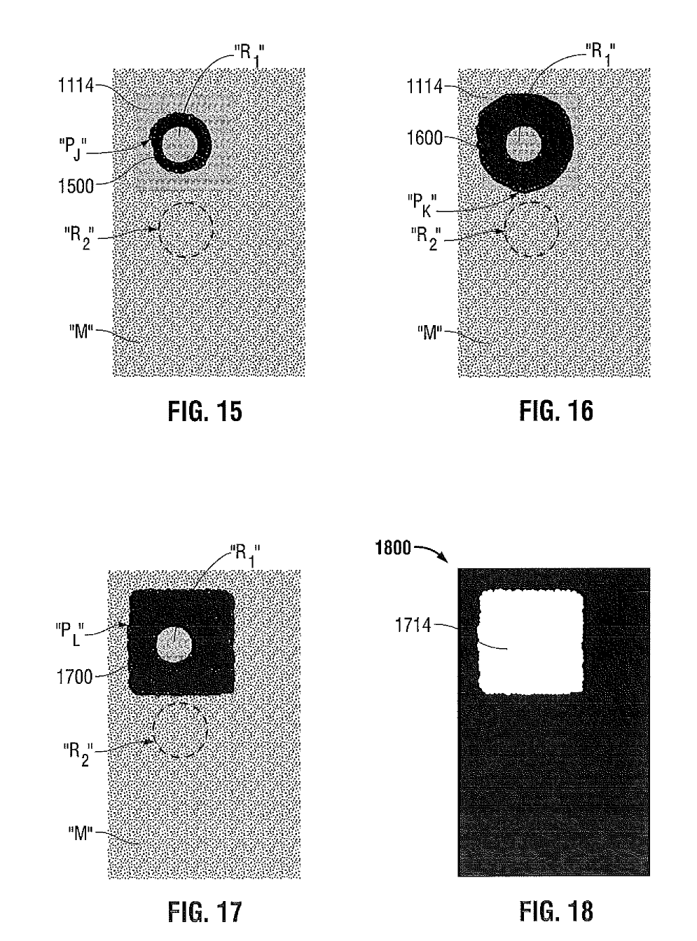

Segmenting an object of interest (e.g., 1114 shown in FIG. 11) from the surrounding medium (e.g., “M” shown in FIGS. 11 through 17) may additionally, or alternatively, involve region growing. Region growing involves multiple iterations to join or not join the examined pixels (e.g., P_{J}, P_{K} and P_{L}, shown in FIGS. 15, 16 and 17, respectively) to the processed entity (e.g., 1500, 1600 and 1700 shown in FIGS. 15, 16 and 17, respectively) at each growth step.

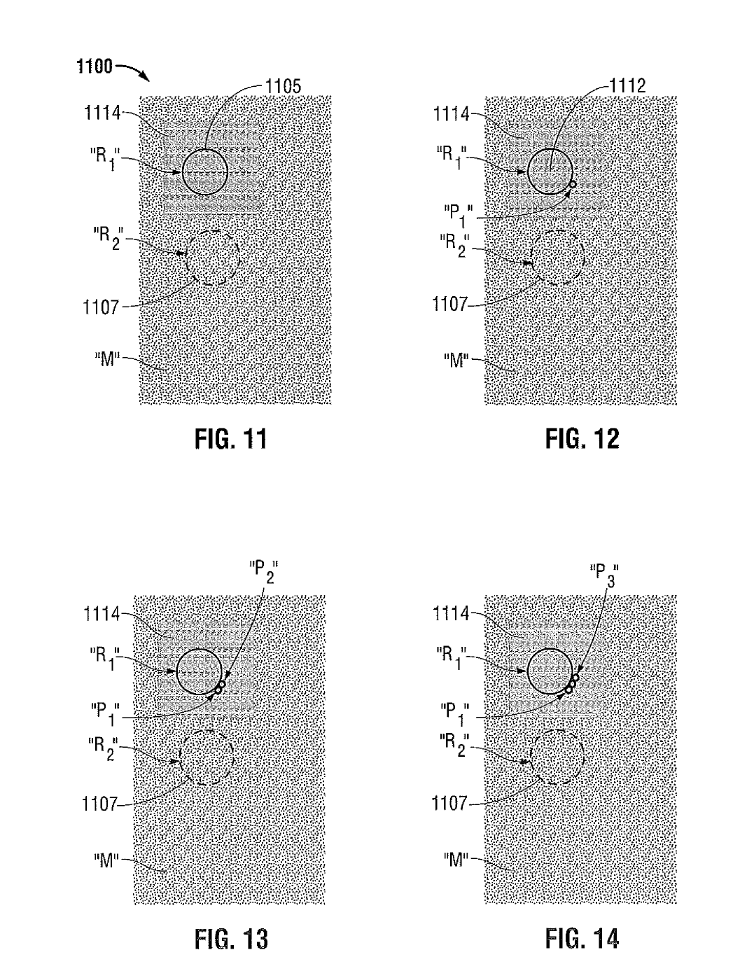

A region-growing method, according to various embodiments, includes the selection and processing of a first pixel or pixel cluster (e.g., “P₁” shown in FIG. 12), followed by the selection and processing of a neighboring, second pixel or pixel cluster (e.g., “P₂” shown in FIG. 13), followed by the selection and processing of a neighboring, third pixel or pixel cluster (e.g., “P₃” shown in FIG. 14), and so on. In various embodiments, the region-growing operations continue so long as there are remaining unexamined pixels within the region of interest (e.g., “R₁” shown in FIG. 17) and/or within the object of interest (e.g., 1114 shown in FIG. 16). When there are no remaining unexamined pixels within the object of interest, e.g., as shown in FIG. 17, thresholding may be performed to obtain a thresholded image (shown generally as 1800 in FIG. 18) of the segmented object of interest (e.g., 1714 shown in FIG. 18). One or more thresholded images may be used to determine the (x,y) coordinates corresponding to the boundaries of the segmented object of interest (e.g., 1714 shown in FIG. 18).

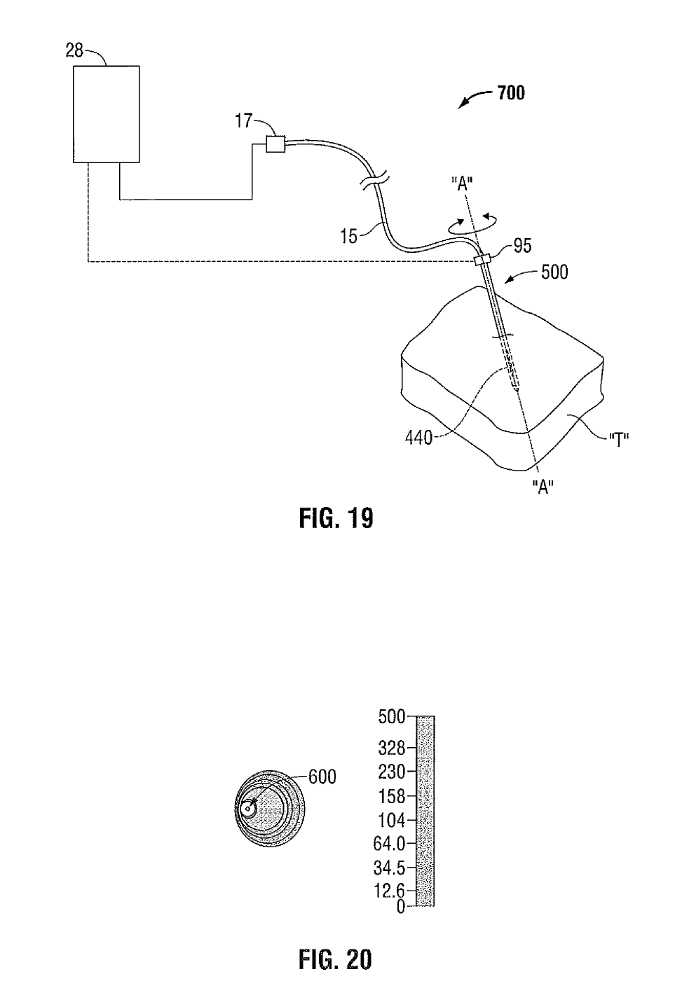

FIG. 19 shows an electrosurgical system 700 according to an embodiment of the present disclosure that includes an ablation device 500 with a directional radiation pattern. Ablation device 500 is coupled to a connector 17 via a transmission line 15, which may further connect the ablation device 500 to an electrosurgical power generating source 28, e.g., a microwave or RF electrosurgical generator. Ablation device 500 includes an elongated body member defining a body wall surrounding a chamber, which is configured to receive at least a portion of an energy applicator therein. The body wall is provided with at least one opening 440 therethough to allow electromagnetic energy radiated from the energy applicator to transfer into a target volume of tissue. In some embodiments, the opening 440 is configured for radiating energy in a broadside radiation pattern, such as the non-limiting example directional radiation pattern shown in FIG. 20. It will be understood, however, that other electrosurgical device embodiments may also be used.

During a procedure, e.g., an ablation procedure, the electrosurgical device 500 of the electrosurgical system 700 is inserted into or placed adjacent to tissue “T” and energy is supplied thereto. Ablation device 500 may be selectively rotated about axis “A-A” (as indicated by the bidirectional arrow in FIG. 19) such that the directional radiation pattern rotates therewith. In embodiments, the ablation device 500 may be selectively rotated about axis “A-A” manually by the user or automatically. An actuator 95 may be operably coupled to the ablation device 500 for controlling the rotation of the ablation device 500 in an automatic process. Actuator 95 may be operably coupled to the electrosurgical power generating source 28 and/or a user interface (e.g., 50 shown in FIG. 1).

In embodiments, the position of an energy applicator may be adjusted based on one or more parameters of a volume-rendered ablation. For example, an energy applicator with a directional radiation pattern, such as the ablation device 500, may be rotated either manually, or automatically, based on one or more parameters of a volume-rendered ablation, e.g., to avoid ablating sensitive structures, such as large vessels, healthy organs or vital membrane barriers. Examples of antenna assemblies rotatable such that any elongated radiation lobes rotates therewith are disclosed in commonly assigned U.S. patent application Ser. No. 12/197,405 filed on Aug. 25, 2008, entitled “MICROWAVE ANTENNA ASSEMBLY HAVING A DIELECTRIC BODY PORTION WITH RADIAL PARTITIONS OF DIELECTRIC MATERIAL”, U.S. patent application Ser. No. 12/535,856 filed on Aug. 5, 2009, entitled “DIRECTIVE WINDOW ABLATION ANTENNA WITH DIELECTRIC LOADING”, and U.S. patent application Ser. No. 12/476,960 filed on Jun. 2, 2009, entitled “ELECTROSURGICAL DEVICES WITH DIRECTIONAL RADIATION PATTERN”.

FIG. 20 is a schematically-illustrated representation of simulation results showing a directional radiation pattern. The illustrated results are based on a simulation that modeled operation of an electrosurgical device 600, which is configured to operate with a directional radiation pattern. Electrosurgical device 600 shown in FIG. 20 is similar to the ablation device 500 of FIG. 19 and further description thereof is omitted in the interests of brevity.

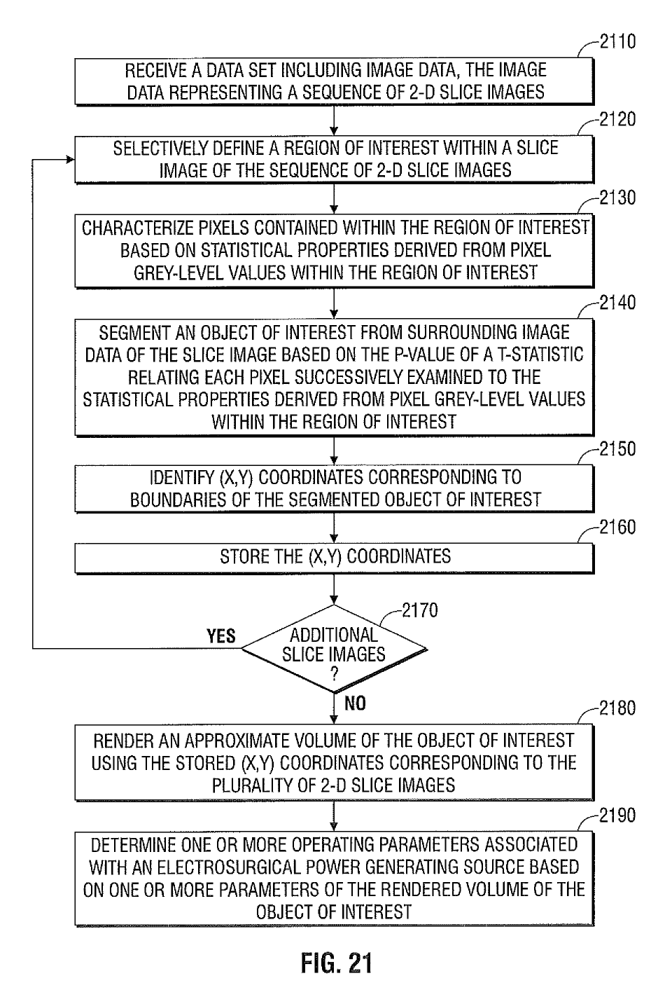

FIG. 21 is a flowchart illustrating a method of image analysis according to an embodiment of the present disclosure. In step 2110, a data set including image data is received. The image data represents a sequence of 2-D slice images, e.g., DICOM format images of any part of the body or a full-body scan. The image data may include inter-operatively acquired images and/or pre-operatively acquired images. A subset of the image data may be selectively defined for processing in accordance with the present method of image analysis illustrated in FIG. 21. For example, the image data may be displayed on a display device (e.g., 21 shown in FIG. 1) and the user may identify a subset of the image data associated with an object of interest (e.g., 1114 shown in FIGS. 11 through 16).

In step 2120, a region of interest is selectively defined within a slice image (e.g., 1100 shown in FIG. 11) of the sequence of 2-D slice images. In FIG. 11, the area “R₁” within the circle 1105 is a user-defined region of interest. Referring to FIG. 1, the clinician may use a pointing device 27 coupled to the processor unit 26 and/or the touchscreen capability of the display device 21 of the electrosurgical system 100 to create a circle, or other shape element, drawn around a selected region of interest (e.g., “R₁” shown in FIG. 11) within an object of interest (e.g., 1114 shown in FIG. 11). Additionally, a non-inclusion region (e.g., “R₂” shown in FIG. 11) may be selectively defined within the object of interest.

In step 2130, pixels contained within the region of interest are characterized based on statistical properties derived from pixel grey-level values within the region of interest. In embodiments, the mean pixel grey-level values and their standard deviations are measured within the region of interest (e.g., “R₁” shown in FIG. 11). In embodiments, the pixel intensity threshold is any pixel in the entire image that is within a mean value of the region-of-interest gray levels+/−a predetermined multiple of the standard deviation of the region-of-interest gray levels. In one embodiment, the predetermined multiple of the standard deviation is 1.8.

In step 2140, an object of interest (e.g., 1114 shown in FIGS. 11 through 16) is segmented from the surrounding image data based on a p-value of a t-statistic relating each pixel successively examined to the statistical properties derived in step 2130. Starting at a seed pixel (e.g., 1112 shown in FIG. 12) or a shape element (e.g., circle 1105 shown in FIG. 12), the presently disclosed method of region growing proceeds to iteratively examine adjacent pixels or pixel clusters (e.g., P_{J}, P_{K} and P_{L}, shown in FIGS. 15, 16 and 17, respectively), and pixels are appended or not appended to the processed entity based on the value of the probability that they are equal to a predetermined probability threshold.

In step 2150, (x,y) coordinates corresponding to the boundaries of the segmented object of interest are determined. The (x,y) coordinates are stored in a memory, in step 2160.

In step 2170, it is determined whether additional slice images remain to be processed. If it is determined that there are additional slice images, then the method repeats step 2120 through step 2160, as described above.

If it is determined that there are no additional slice images, then, in step 2180, an approximate volume of the object of interest is rendered using the stored (x,y) coordinates corresponding to the plurality of 2-D slice images. Least-squares fitting (regression) may be used in the volume-rendering process.

In step 2190, one or more operating parameters associated with the electrosurgical power generating source (e.g., 16 shown in FIG. 1) are determined based one or more parameters of a rendered volume of the object of interest. Examples of operating parameters associated with the electrosurgical power generating source include without limitation temperature, impedance, power, current, voltage, mode of operation, and duration of application of electromagnetic energy. Examples of parameters of a volume-rendered object of interest (e.g., 700 and 800 shown in FIGS. 7 and 8, respectively) include without limitation volume, length, diameter, minimum diameter, maximum diameter and centroid.

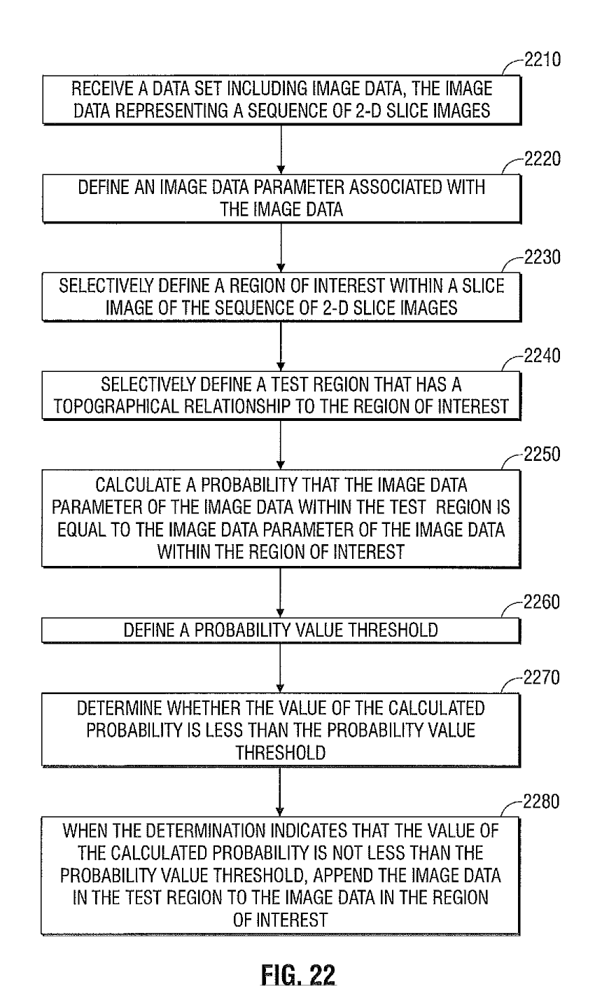

FIG. 22 is a flowchart illustrating a method of image analysis according to an embodiment of the present disclosure. In step 2210, a data set including image data is received. The image data represents a sequence of 2-D slice images.

In step 2220, an image-data parameter associated with the image data is defined. In some embodiments, the image-data parameter is pixel grey-level value. In cases where the image data includes color image data and/or multiple images of an area acquired with multiple imaging modalities, the image-data parameter may be a vector distance.

In step 2230, a region of interest (e.g., “R₁” shown in FIG. 11) is selectively defined (e.g., circle 1105 shown in FIG. 11) within a slice image (e.g., 1100 shown in FIG. 11) of the sequence of 2-D slice images. The region of interest may be automatically selected, e.g., using knowledge of an anatomical structure. The region of interest may be user-defined. Additionally, a non-inclusion region (e.g., “R₂” shown in FIG. 11) may be selectively defined (e.g., dashed circle 1107 shown in FIG. 11).

In step 2240, a test region (e.g., “P₁” shown in FIG. 12) is selectively defined that has a topographical relationship to the region of interest. In some embodiments, the topographical relationship is about the perimeter of the region of interest (e.g., “R₁” shown in FIG. 11).

In step 2250, a probability is calculated that the image-data parameter of the image data within the test region is equal to the image-data parameter of the image data within the region of interest.

In step 2260, a probability value threshold is defined. The probability value threshold may be any suitable value. In some embodiments, the probability value threshold is 0.95.

In step 2270, determine whether the value of the probability calculated in step 2250 (also referred to herein as the calculated probability) is less than the probability value threshold.

In step 2280, when the determination indicates that the value of the calculated probability is not less than the probability value threshold, append the image data within the test region to the image data within the region of interest.

The above-described electrosurgical systems and methods of directing electromagnetic radiation to tissue according to embodiments of the present disclosure may allow clinicians to avoid ablating or unnecessarily heating tissue structures, such as large vessels, healthy organs or vital membrane barriers, by adjusting the ablation field radiating into tissue based on one or more parameters of a volume-rendered ablation that is generated from image data representing a sequence of 2-D slice images. A real-time or near real-time volume rendering process may allow clinicians to visualize the ablative process while it is occurring.

The above-described electrosurgical systems may enable a user to view one or more ablation patterns and/or other energy applicator data corresponding to an embodiment of an ablation device, which may allow clinicians to predict ablation volume, avoid complications, and/or plan for treatment margins.

It is envisioned and within the scope of the present disclosure that image data associated with a prior treatment of a target tissue volume may be retrieved from a database and used in accordance with the above-described methods of image analysis to generate a volume-rendered ablation. Information from the volume-rendered ablation may be used by clinicians during the pre-operative stage of a medical procedure.

The above-described methods of image analysis may be suitable for use in surgical or non-surgical (e.g., interventional radiology, etc.) settings.

Although embodiments have been described in detail with reference to the accompanying drawings for the purpose of illustration and description, it is to be understood that the inventive processes and apparatus are not to be construed as limited thereby. It will be apparent to those of ordinary skill in the art that various modifications to the foregoing embodiments may be made without departing from the scope of the disclosure.

What is claimed is:

1. A method of image analysis, comprising the steps of:
receiving a data set including image data, the image data representing a sequence of two-dimensional (2-D) slice images;
segmenting an object of interest from surrounding image data of each slice image based on a p-value of a t-statistic relating each pixel successively examined to statistical properties derived from pixel values within the region of interest; and
rendering a volume of the object of interest using (x,y) coordinates corresponding to boundaries of the segmented object of interest.

2. The method of image analysis in accordance with claim 1, wherein rendering the volume of the object of interest using (x,y) coordinates corresponding to boundaries of the segmented object of interest includes the steps of:
determining (x,y) coordinates corresponding to boundaries of the segmented object of interest of each slice image;
arranging the boundaries of the segmented object of interest of each slice image along a third dimension using the (x,y) coordinates corresponding to the plurality of 2-D slice images; and
fitting a 3-D surface to the arranged boundaries to render a volume of the object of interest.

3. The method of image analysis in accordance with claim 1, wherein segmenting an object of interest from surrounding image data of each slice image based on a topographic growth rule and a pixel intensity threshold value with respect to a starting pixel includes the steps of:
selectively defining a region of interest within each slice image of the sequence of 2-D slice images; and
characterizing pixels contained within the region of interest of each slice image based on statistical properties derived from pixel values within the region of interest of each slice image.

4. The method of image analysis in accordance with claim 3, wherein the statistical properties include mean pixel grey-level values and standard deviations of the region of interest gray levels.

5. The method of image analysis in accordance with claim 1, wherein the image data representing the sequence of 2-D slice images is in DICOM format.

6. The method of image analysis in accordance with claim 1, further comprising the step of:
displaying the rendered volume of the object of interest on a display device to facilitate planning of a procedure.

7. The method of image analysis in accordance with claim 1, further comprising the step of:
determining at least one operating parameter associated with an electrosurgical power generating source based on at least one parameter of the rendered volume of the object of interest.

8. The method of image analysis in accordance with claim 7, wherein the at least one parameter of the rendered volume of the object of interest is selected from the group consisting of volume, length, diameter, minimum diameter, maximum diameter and centroid.

9. A method of image analysis, comprising the steps of:
receiving a data set including image data, the image data representing a sequence of two-dimensional (2-D) slice images;
selectively defining a region of interest within each slice image of the sequence of 2-D slice images;
characterizing pixels contained within the region of interest of each slice image based on statistical properties derived from pixel values within the region of interest of each slice image;
segmenting an object of interest from surrounding image data of each slice image based on a p-value of a t-statistic relating each pixel successively examined to statistical properties derived from pixel values within the region of interest;
determining (x,y) coordinates corresponding to boundaries of the segmented object of interest of each slice image; and
rendering a volume of the object of interest using the (x,y) coordinates corresponding to the plurality of 2-D slice images.

10. The method of image analysis in accordance with claim 9, wherein the statistical properties include mean pixel values and their standard deviations.

11. The method of image analysis in accordance with claim 9, wherein the step of selectively defining a region of interest within each slice image of the sequence of 2-D slice images includes selecting a starting pixel within each slice image.

12. The method of image analysis in accordance with claim 9, further comprising the step of:
determining at least one operating parameter associated with an electrosurgical power generating source based on at least one parameter of the rendered volume of the object of interest.

13. The method of image analysis in accordance with claim 9, wherein the step of receiving a data set including image data includes retrieving image data from a picture archiving and communication system (PACS).

14. The method of image analysis in accordance with claim 9, further comprising the step of:
displaying the rendered volume of the object of interest on a display device to facilitate planning of a procedure.

15. The method of image analysis in accordance with claim 9, wherein selectively defining the region of interest includes the steps of:
displaying image data on a display device; and
providing a pointing device to enable user selection of the region of interest.

16. An electrosurgical system, comprising:
an electrosurgical power generating source;
an energy-delivery device operably associated with the electrosurgical power generating source;
a processor unit; and
an imaging system capable of generating image data representing a sequence of 2-D slice images,
wherein the processor unit is disposed in operative communication with the imaging system and adapted to analyze the image data to segment an object of interest from surrounding image data of each slice image based on a p-value of a t-statistic relating each pixel successively examined to statistical properties derived from pixel values within the region of interest.

17. The electrosurgical system of claim 16, wherein the processor unit is further adapted to render a volume of the object of interest using (x,y) coordinates corresponding to boundaries of the segmented object of interest.

18. The electrosurgical system of claim 16, wherein the energy-delivery device is configured to emit a directional radiation pattern that rotates therewith during rotation of the energy-delivery device about a longitudinal axis thereof.

19. The electrosurgical system of claim 18, wherein the processor unit is further adapted to control rotation of the energy-delivery device about the longitudinal axis thereof during a treatment procedure based on at least one parameter of the rendered volume of the object of interest.
